(19) United States
(12) Patent Application Publication
(10) Pub. No.: US 2025/0312568 A1
(43) Pub. Date: Oct. 9, 2025

(54) CATHETER ASSEMBLY WITH MINIATURE DEVICE

(71) Applicant: BIONAUT LABS LTD., Herzliya (IL)

(72) Inventors: Florent CROS, Los Angeles, CA (US); Alex KISELYOV, San Diego, CA (US); Jason SHIH, Pasadena, CA (US)

(73) Assignee: BIONAUT LABS LTD., Herzliya (IL)

(21) Appl. No.: 19/172,555

(22) Filed: Apr. 7, 2025

Related U.S. Application Data
(60) Provisional application No. 63/631,650, filed on Apr. 9, 2024.

(51) Int. Cl. A61M 25/01; A61M 25/00

(52) U.S. Cl. CPC A61M 25/0127; A61M 25/0069; A61M 25/0147; A61M 2202/0007; A61M 2205/0216; A61M 2205/50; A61M 2205/82

(57) ABSTRACT

The presently disclosed subject matter provides catheter assemblies configured for being maneuvered within a patient toward a treatment site, the catheter assembly comprising: a catheter comprising an elongated flexible shaft and having a distal end for introduction into the patient; and a miniature device configured to be attached to the distal end of the catheter and to be maneuvered within the patient in response to an external non-contact force, the miniature device further being configured to selectively detach from the catheter within the patient and to be maneuvered in response to the external non-contact force when detached from the catheter; wherein maneuvering of the miniature device when attached to the catheter facilitates maneuvering of the catheter within the patient.

### CROSS REFERENCE TO RELATED APPLICATIONS

This application claims the benefit of priority of U.S. Provisional Application No. 63/631,650, filed Apr. 9, 2024, hereby incorporated by reference.

### TECHNOLOGICAL FIELD

The presently disclosed subject matter relates to systems and miniature devices configured to navigate within a patient to perform a therapeutic activity at a predetermined target site therewithin, and in particular to such systems which use magnetic fields to direct operation of miniature devices within a patient.

### BACKGROUND

Remote control of miniature medical devices inside the human body can be useful for a variety of purposes, including, but not limited to, delivery of therapeutic payloads, diagnostics, and surgical procedures. Such devices may include microscale or nanoscale robots, medical tools, “smart pills,” etc. The most suitable type of motion, e.g., forward, screwing, etc., may depend on the environment, e.g., including the type of matter, in which the miniature device is situated.

### SUMMARY

According to an aspect of the presently disclosed subject matter, there is provided a catheter assembly configured for being maneuvered within a patient toward a treatment site, the catheter assembly comprising:
a catheter comprising an elongated flexible shaft and having a distal end for introduction into the patient; and
a miniature device configured to be attached to the distal end of the catheter and to be maneuvered within the patient in response to an external non-contact force, the miniature device further being configured to selectively detach from the catheter within the patient and to be maneuvered in response to the external non-contact force when detached from the catheter;
   wherein maneuvering of the miniature device when attached to the catheter facilitates maneuvering of the catheter within the patient.

The external non-contact force may comprise a magnetic force.

The catheter and the miniature device may be configured to communicate with each other, at least when the miniature device is attached to the catheter.

The catheter may be configured to supply power to the miniature device, at least when the miniature device is attached to the catheter.

The catheter may be configured to supply one or more materials to the miniature device, at least when the miniature device is attached to the catheter.

The miniature device may be configured to provide one or more materials collected from the patient to the catheter.

The catheter assembly may further comprise a tether connected to the miniature device and the catheter, the tether being configured to remain attached to the miniature device and the catheter when the miniature device detaches from the catheter.

The catheter and the miniature device may be configured to communicate via the tether, at least when the miniature device is detached from the catheter.

The catheter may be configured to supply power to the miniature device via the tether, at least when the miniature device is detached from the catheter.

The catheter may be configured to supply one or more materials to the miniature device via the tether, at least when the miniature device is detached from the catheter.

The catheter may be configured to provide one or more materials collected from the patient to the catheter via the tether, at least when the miniature device is detached from the catheter.

The miniature device may be configured to detach from the catheter in response to an auxiliary non-contact force.

The auxiliary non-contact force may comprise a magnetic force.

One of the non-contact force and the auxiliary non-contact force may be produced by a linear magnetic field, with the other of the non-contact force and the auxiliary non-contact force being produced by a rotating magnetic field.

The miniature device may be configured to selectively reattach to the distal end of the catheter.

The miniature device may be configured to reattach to the distal end of the catheter in response to an auxiliary non-contact force.

The catheter may be configured to be maneuvered independently of the response of the miniature device to the external non-contact force.

The catheter assembly may further comprise a pull-wire system configured to further facilitate maneuvering of the catheter.

The pull-wire system may comprise one or more pull-wires each extending longitudinal along at least a portion of the shaft, the shaft being configured to be deflected when a longitudinal tension is applied to each of the pull-wires.

The catheter assembly may further comprise a pressurized-fluid system to further facilitate maneuvering of the catheter.

The pressurized-fluid system may be a pneumatic system.

The pressurized-fluid system may be a hydraulic system.

The catheter assembly may further comprise one or more mechanical actuators, separate from the miniature device, configured to selectively deflect the distal end of the catheter.

The miniature device may be configured to perform a medical activity within the patient, at least when detached from the catheter.

The medical activity may comprise delivering one or more medical materials.

The one or more medical materials may comprise a therapeutic and/or a diagnostic agent.

The one or more medical materials may comprise at least one selected from the group consisting of a one or more chemical compounds, one or more small molecules, one or more biologics, one or more cells, one or more endogenous cells, one or more engineered cell, one or more radioisotopes, one or more vaccines, a microfluid, one or more peptides, one or more peptoids, one or more oligonucleotide sequences, one or more nucleic acids, and one or more oncolytic viruses.

The medical activity may comprise performing a medical procedure inside the patient.

The medical activity may comprise performing a microsurgical procedure.

The medical activity may comprise a neurostimulation and/or a neurodiagnostic procedure.

The miniature device may be configured to form a channel within solid tissue of the patient.

The catheter may be hollow, defining one or more lumens passing therethrough.

At least one of the lumens may be sized so as to permit passage therethrough of the miniature device.

According to another aspect of the presently disclosed subject matter, there is provided a system comprising:
the catheter assembly as described above;
a driving arrangement configured to produce one or more magnetic fields; and
a controller configured to direct operation of the driving arrangement to produce the magnetic fields, wherein the produced magnetic fields are configured to maneuver the miniature device of the catheter assembly.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates a catheter assembly according to the presently disclosed subject matter, in which a miniature device thereof is attached to a catheter thereof;

FIG. 2 illustrates the catheter assembly of FIG. 1, in which the miniature device is detached from the catheter; and

FIG. 3 illustrates the catheter assembly of FIG. 1, in which the miniature device is detached from the catheter and connected thereto via a tether.

### DETAILED DESCRIPTION OF EMBODIMENTS

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

According to the presently disclosed subject matter, there is provided a system for facilitating performing a medical activity at a treatment site in a patient.

The system comprises a catheter assembly and a driving arrangement configured to produce one or more magnetic fields. It further comprises a controller configured to direct operation of the other elements of the system. In particular, the controller is configured to produce one or more magnetic fields, including varying them as necessary, in order to maneuver parts of the catheter assembly, as will be described below.

It will be appreciated that while herein the specification and claims, the term “controller” is used with reference to a single element, it may in practice comprise a plurality of elements, which may or may not be in physical proximity to one another, without departing from the scope of the presently disclosed subject matter, mutatis mutandis. In addition, disclosure herein (including recitation in the appended claims) of a controller carrying out, being configured to carry out, or other similar language, implicitly includes other elements of the system carrying out, being configured to carry out, etc., those functions, without departing from the scope of the presently disclosed subject matter, mutatis mutandis.

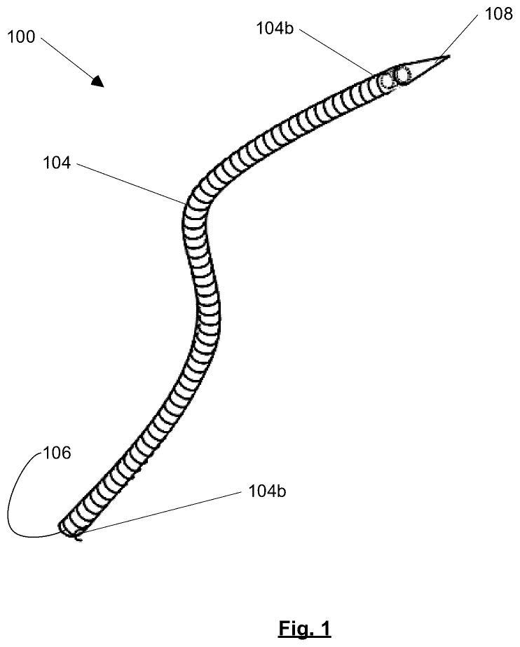

As illustrated in FIG. 1, the catheter assembly 100 comprises a catheter 104 being formed as an elongated flexible shaft having a proximal end 104a and a distal end 104b for introduction into the patient. The catheter 104 may be hollow, for example defining a lumen 106 therethrough. It will be appreciated that while the specification and appended claims refer to a catheter, any suitable elongated flexible shaft, optionally defining a lumen therethrough, e.g., a cannula, may be provided without departing from the scope of the presently disclosed subject matter, mutatis mutandis.

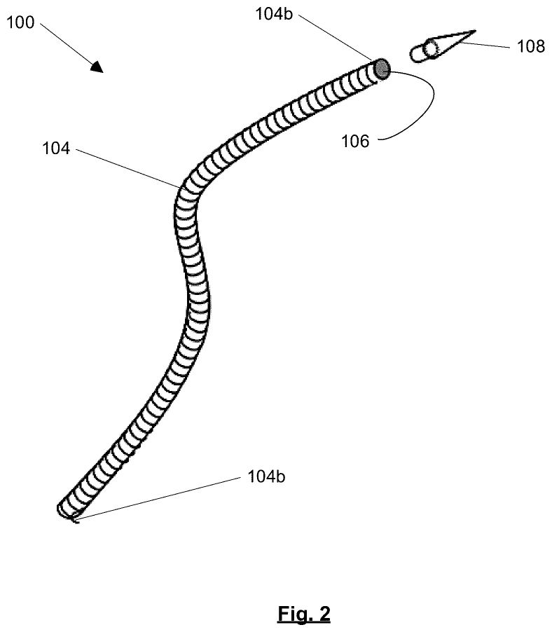

The catheter assembly 100 further comprises a miniature device 108, configured to perform the medical activity at the treatment site. The miniature device is further configured to be attached to the catheter 104, for example at (i.e., at or near) its distal end 104b, and further configured to be detached therefrom and to move independently of the catheter once detached, for example as illustrated in FIG. 2. The attachment of the miniature device to the catheter 104 may be accomplished by any suitable means, for example mechanically, electromagnetically, etc. Accordingly, the miniature device may be configured to perform the medical activity when detached from and/or when attached to the catheter 108.

According to some examples, the medical activity comprises delivery of one or more medical materials to the treatment site. The medical materials may comprise a therapeutic and/or diagnostic agent. It may comprise one or more chemical compounds, one or more small molecules, one or more biologics, one or more cells, one or more endogenous cells, one or more engineered cell, one or more radioisotopes, one or more vaccines, a microfluid, one or more peptides, one or more peptoids, one or more oligonucleotide sequences, one or more nucleic acids, and/or one or more oncolytic viruses, etc., of medicinal, diagnostic, evaluative, and/or therapeutic relevance. According to some examples, the treatment site is in the patient's brain and/or one or more other areas of the central nervous system.

According to some examples, the medical activity comprises performing a medical procedure at the treatment site. The medical procedure may comprise performing a microsurgical procedure, a neurostimulation and/or a neurodiagnostic procedure, or any other suitable procedure.

According to some examples, the medical activity comprises forming a channel within solid tissue of the patient. In this connection, the miniature device 108 may be, mutatis mutandis, provided and configured to operate as per that disclosed in co-pending international patent application number PCT/US24/21538, filed on Mar. 26, 2024, relevant portions of which are incorporated herein by reference.

It will be appreciated that herein the specification and appended claims, the term “treatment site” may be used to refer to the location where the system acts, even if the location which is treated is located elsewhere. For example, the system may be configured to perform a cutting operation at a location in order to access an adjacent location to deliver a medical material thereto. In such a case, the location which undergoes the cutting operation may be referred to herein as the “treatment site,” even though it is not the ultimate recipient of the medical material. In some cases, both the location at which the cutting operation is performed, as well as the location which is the ultimate recipient of the medical materials, may each be considered a “treatment site.”

The miniature device 108 is configured to be remotely maneuvered by an externally applied primary non-contact force, for example produced by a magnetic field having parameters selected to cause the miniature device to undergo a predetermined motion (e.g., forward motion, rotational motion, etc.). The parameters may include, but are not limited to, strength, direction, rate of variation, etc. In order for the miniature device 108 to perform complex motions, e.g., traveling along a non-linear path, etc., the parameters may be continuously adjusted, e.g., according to a pre-determined program, based on feedback received regarding its location, etc.

In particular, the catheter assembly 100 is configured such that when the miniature device 108 is attached to the catheter 104, maneuvering of the catheter within the patient, for example toward the treatment site, is facilitated by the motion of the miniature device as it is maneuvered within the patient. This may include, but is not limited to, motion of the miniature device 108 guiding the direction of the catheter 104, e.g., by facilitating deflection of its distal end 104b, the miniature device pulling the catheter as it moves within the patient, etc.

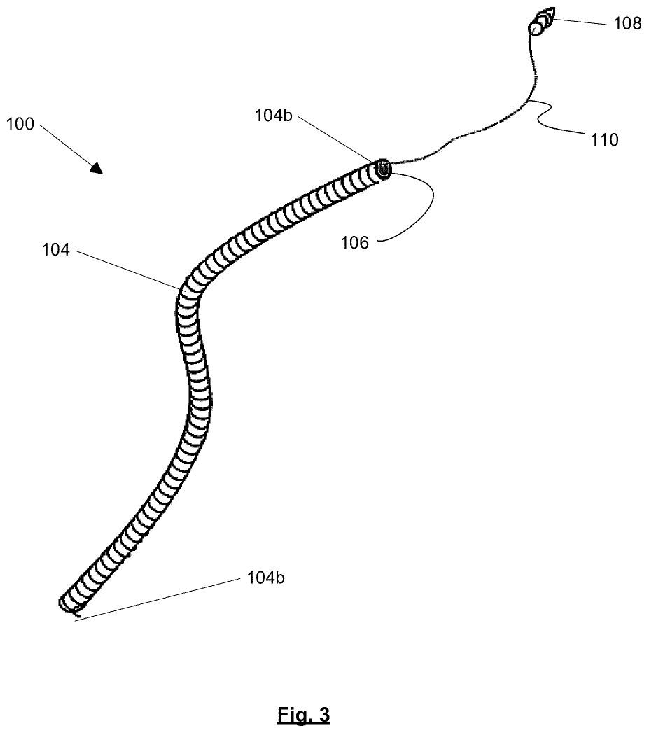

As illustrated in FIG. 3, when the miniature device 108 is detached from the catheter 104, it may remain connected thereto by a tether 110 which is connected to the miniature device and catheter.

The catheter 104 and the miniature device 108 may be configured to communicate with each other. It will be appreciated that references herein the specification and appended claims of communication with the catheter 104 includes communication via the catheter, i.e., the miniature device may be in communication with an external system, for example the controller of the system, via the catheter.

The communication may comprise supplying power, e.g., electrical power, to the miniature device 108. The communication may comprise transmission of data between the catheter and the miniature device 108.

According to some examples, the catheter 104 and miniature device 108 may each comprise an electrical contact (not illustrated), wherein the catheter and miniature device are configured such that when the miniature device is attached to the catheter their electrical contacts are in contact with each other, thereby facilitating transmission of power and/or data via electronic communication.

According to other examples, the catheter 104 and the miniature device 108 may each comprise suitable components to facilitate wireless power transmission and/or wireless data transmission, for example as is well known in the art. While wireless communication with the miniature device 108 may be accomplished from outside the patient, it may be advantageous to communicate with the miniature device with the catheter 104, for example to take advantage of its proximity thereto.

The communication may further comprise supplying materials to the miniature device 108 and/or providing materials, e.g., collected from the patient by the miniature device, to the catheter 104. In the latter case, the catheter assembly 100 may be used to perform a medical procedure related to diagnosis of the patient, monitoring another medical procedure, etc.

According to some examples, the tether 110 is configured to facilitate communication between the catheter 104 and the miniature device 108. Accordingly, the tether 110 may comprise an electrical conducting element spanning its length, e.g., to facilitate power supply and/or data transmission, or be formed with a throughgoing lumen, e.g., to facilitate movement of materials between the catheter and the miniature device.

The miniature device 108 may be configured to detach from and/or reattach to the catheter 104 based on one or more predetermined condition. According to different examples, the miniature device 108, the catheter 104, or a combination of both may be responsive to the predetermined condition to effect the detaching/attaching.

The predetermined condition may include, but is not limited to, an environmental condition (e.g., temperature, pH, detection of one or more predetermined chemicals, etc.) or combination thereof, the passage of a predetermined amount of time, an external signal applied wirelessly and/or via the catheter 104—optionally via the tether 110, application of an auxiliary non-contact force (i.e., separate from the primary non-contact force which maneuvers the miniature device), etc.

According to some examples, the auxiliary non-contact force comprises a magnetic force. In order to facilitate distinguishing between the primary non-contact force applied to maneuver the miniature device 108, and the auxiliary non-contact force applied to facilitate detaching/attaching of the miniature device from/to the catheter 104, one of the non-contact forces, e.g., the primary non-contact force, may be produced by a linear magnetic field, while the other of the non-contact forces, e.g., the auxiliary non-contact force, may be produced by a rotating magnetic field.

According to some examples, detaching/attaching of the miniature device 108 from/to the catheter 104 is accomplished by maneuvering the miniature device, e.g., with the primary non-contact force. For example, the distal end 104b of the catheter 104 and the miniature device 108 may be formed with cooperating parts constituting a bayonet mount (not illustrated), whereby detaching/attaching of the miniature device 108 may be accomplished by suitably maneuvering the miniature device 108.

The catheter 104 may be configured to be maneuvered independently of the response of the miniature device 108 to the primary non-contact force, i.e., the catheter may be configured to be maneuvered independent of the motion of the miniature device.

According to some examples, the catheter assembly 100 may comprise a pull-wire system, for example as is known in the art, configured to facilitate maneuvering of the catheter 104 independent of the motion of the miniature device. The pull-wire system may be configured to be operated manually by an operator and/or by the controller. The pull-wire system may comprise one or more pull-wires extending longitudinally along at least a portion of the flexible shaft of the catheter 104. The catheter is configured to be deflected when a longitudinal tension is applied to each of the pull-wires, thereby facilitating maneuvering thereof.

According to some examples, the catheter assembly 100 may comprise a pressurized-fluid system, for example as is known in the art, configured to facilitate maneuvering of the catheter 104 independent of the motion of the miniature device. The pressurized-fluid system may comprise a pneumatic system and/or a hydraulic system.

According to some examples, the catheter assembly 100 may comprise one or more mechanical actuators, for example as is known in the art, separate from the miniature device 108 and being configured to facilitate maneuvering of the catheter 104 independent of the motion of the miniature device. According to some examples, the mechanical actuators are configured to facilitate maneuvering of the catheter 104 by selectively deflecting its distal end 104b.

The independent maneuvering of the catheter 104 may be in addition to or an alternative to the catheter assembly 100 being configured such that the motion of the miniature device 108 as it is maneuvered within the patient facilitates maneuvering of the catheter, for example as described above.

According to some examples, the catheter 104 is configured to facilitate delivery of additional miniature devices to the treatment site, for example once it has been maneuvered to a location within the patient. Accordingly, it may be formed with a lumen which is sized so as to permit passage therethrough of the additional miniature devices, e.g., which may be the same size as the miniature device 108.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A catheter assembly configured for being maneuvered within a patient toward a treatment site, the catheter assembly comprising:
a catheter comprising an elongated flexible shaft and having a distal end for introduction into the patient; and
a miniature device configured to be attached to the distal end of the catheter and to be maneuvered within the patient in response to an external non-contact force, the miniature device further being configured to selectively detach from the catheter within the patient and to be maneuvered in response to the external non-contact force when detached from the catheter;
wherein maneuvering of the miniature device when attached to the catheter facilitates maneuvering of the catheter within the patient.

2. The catheter assembly according to claim 1, wherein the external non-contact force comprises a magnetic force.

3. The catheter assembly according to claim 1, wherein the catheter and the miniature device are configured to communicate with each other, at least when the miniature device is attached to the catheter.

4. The catheter assembly according to claim 1, wherein the catheter is configured to supply power to the miniature device, at least when the miniature device is attached to the catheter.

5. The catheter assembly according to claim 1, wherein the catheter is configured to supply one or more materials to the miniature device, at least when the miniature device is attached to the catheter.

6. The catheter assembly according to claim 1, wherein the miniature device is configured to provide one or more materials collected from the patient to the catheter.

7. The catheter assembly according to claim 1, further comprising a tether connected to the miniature device and the catheter, the tether being configured to remain attached to the miniature device and the catheter when the miniature device detaches from the catheter.

8. The catheter assembly according to claim 7, wherein the catheter and the miniature device are configured to communicate via the tether, at least when the miniature device is detached from the catheter.

9. The catheter assembly according to claim 7, wherein the catheter is configured to supply power to the miniature device via the tether, at least when the miniature device is detached from the catheter.

10. The catheter assembly according to claim 7, wherein the catheter is configured to supply one or more materials to the miniature device via the tether, at least when the miniature device is detached from the catheter.

11. The catheter assembly according to claim 7, wherein the catheter is configured to provide one or more materials collected from the patient to the catheter via the tether, at least when the miniature device is detached from the catheter.

12. The catheter assembly according to claim 1, wherein the miniature device is configured to detach from the catheter in response to an auxiliary non-contact force.

13. The catheter assembly according to claim 12, wherein the auxiliary non-contact force comprises a magnetic force.

14. The catheter assembly according to claim 13, wherein one of the non-contact force and the auxiliary non-contact force is produced by a linear magnetic field, and the other of the non-contact force and the auxiliary non-contact force is produced by a rotating magnetic field.

15. The catheter assembly according to claim 1, wherein the miniature device is configured to selectively reattach to the distal end of the catheter.

16. The catheter assembly according to claim 15, wherein the miniature device is configured to reattach to the distal end of the catheter in response to an auxiliary non-contact force.

17. The catheter assembly according to claim 1, wherein the catheter is configured to be maneuvered independently of the response of the miniature device to the external non-contact force.

18. The catheter assembly according to claim 17, further comprising a pull-wire system configured to further facilitate maneuvering of the catheter.

19. The catheter assembly according to claim 18, the pull-wire system comprising one or more pull-wires each extending longitudinal along at least a portion of the shaft, the shaft being configured to be deflected when a longitudinal tension is applied to each of the pull-wires.

20. The catheter assembly according to claim 17, further comprising a pressurized-fluid system to further facilitate maneuvering of the catheter.

21. The catheter assembly according to claim 20, wherein the pressurized-fluid system is a pneumatic system.

22. The catheter assembly according to claim 20, wherein the pressurized-fluid system is a hydraulic system.

23. The catheter assembly according to claim 17, further comprising one or more mechanical actuators, separate from the miniature device, configured to selectively deflect the distal end of the catheter.

24. The catheter assembly according to claim 1, wherein the miniature device is configured to perform a medical activity within the patient, at least when detached from the catheter.

25. The catheter assembly according to claim 24, wherein the medical activity comprises delivering one or more medical materials.

26. The catheter assembly according to claim 25, wherein the one or more medical materials comprises a therapeutic and/or a diagnostic agent.

27. The catheter assembly according to claim 24, wherein the one or more medical materials comprises at least one selected from the group consisting of a one or more chemical compounds, one or more small molecules, one or more biologics, one or more cells, one or more endogenous cells, one or more engineered cell, one or more radioisotopes, one or more vaccines, a microfluid, one or more peptides, one or more peptoids, one or more oligonucleotide sequences, one or more nucleic acids, and one or more oncolytic viruses.

28. The catheter assembly according to claim 24, wherein the medical activity comprises performing a medical procedure inside the patient.

29. The catheter assembly according to claim 28, wherein the medical procedure comprises performing a microsurgical procedure.

30. The catheter assembly according to claim 28, wherein the medical procedure comprises a neurostimulation and/or a neurodiagnostic procedure.

31. The catheter assembly according to claim 1, wherein the miniature device is configured to form a channel within solid tissue of the patient.

32. The catheter assembly according to claim 1, wherein the catheter is hollow, defining one or more lumens passing therethrough.

33. The catheter assembly according to claim 32, wherein at least one of the lumens is sized so as to permit passage therethrough of the miniature device.

34. A system comprising:
the catheter assembly according to claim 1;
a driving arrangement configured to produce one or more magnetic fields; and
a controller configured to direct operation of the driving arrangement to produce the magnetic fields, wherein the produced magnetic fields are configured to maneuver the miniature device of the catheter assembly.
